(19) United States
(12) Patent Application Publication
(10) Pub. No.: US 2026/0337684 A1
(43) Pub. Date: Oct. 29, 2026

(54) COMPOSITION FOR PREDICTING SULFONYLUREA DEPENDENCE BY USING THADA MARKER, AND PREDICTION METHOD THEREFOR

(71) Applicant: Seoul National University Hospital, Seou (KR)

(72) Inventor: Hye Seung Jung, Seoul (KR)

(21) Appl. No.: 19/119,767

(22) PCT Filed: Sep. 27, 2023
(86) PCT No.: PCT/KR2023/015065

§ 371 Date: Apr. 10, 2025

(30) Foreign Application Priority Data
Oct. 12, 2022 (KR) 10-2022-0130239
Sep. 20, 2023 (KR) 10-2023-0125551

(51) Int. Cl. C12Q 1/6883

(52) U.S. Cl. CPC C12Q 1/6883; C12Q 2600/156

(57) ABSTRACT

The present invention relates to a composition and method for predicting sulfonylurea (SU) dependency in diabetes mellitus patients, comprising: An agent that identifies at least one first mutation selected from the group consisting of mutations at the 43,801,878th base (allele A), the 43,801,909th base (allele T), the 43,776,463rd base (allele C), the 43,625,187th base (allele C), the 43,520,160th base (allele C), and the 43,805,692nd base (allele G) in chromosome 2 of THADA (THADA armadillo repeat containing) in GRCh37/hg19; or A second mutation that reduces the function of the THADA protein and has an allele frequency of 5% or less.

### TECHNICAL FIELD

The present invention relates to a composition for predicting sulfonylurea dependency and a method for predicting sulfonylurea dependency.

### BACKGROUND

Compared to research on markers related to susceptibility to the onset of diabetes mellitus, studies on markers related to the effects of diabetes mellitus treatments are minimal. Thus, there are no competing technologies for genetic markers with clinical significance concerning the use of diabetes mellitus treatments, and prescriptions are currently based on the experience of medical professionals.

Currently, nine classes of medications are used for diabetes mellitus treatment, and prescription guidelines by most related societies are not based on predictions of blood glucose-lowering effects or do not contribute to such predictions (Standards of Medical Care in Diabetes, Diabetes Care January 2022; Korean Diabetes Association Diabetes Clinical Practice Guidelines 2021).

Sulfonylureas are a class of highly potent blood glucose-lowering agents with the longest history of use. However, compared to other classes of medications, they are associated with a higher risk of hypoglycemia. Furthermore, the development of other oral blood glucose-lowering agents with proven cardiovascular protective effects has resulted in a decline in the overall prescription priority of sulfonylureas. Nonetheless, certain patient groups heavily rely on sulfonylureas for blood glucose control. For these specific subset of patients, the preferential use of sulfonylureas is necessary, but no clinical characteristics or markers predicting such dependency have been identified to date.

Therefore, there is a need to discover markers related to sulfonylurea dependency for diabetes mellitus treatment and to develop technologies that can identify sensitivity to sulfonylureas based on these markers.

### DETAILED DESCRIPTION OF THE INVENTION

### Technical Problem

The present invention aims to identify genes that determine sulfonylurea dependency and to provide a composition for predicting sulfonylurea dependency.

The present invention also aims to provide a method for offering information to predict sulfonylurea dependency.

### Technical Solution

1. A composition for predicting sulfonylurea dependency in diabetes mellitus patients, comprising an agent that identifies at least one of the following:
A first mutation selected from the group consisting of mutations of the 43801878th base (allele A), the 43801909th base (allele T), the 43776463rd base (allele C), the 43625187th base (allele C), the 43520160th base (allele C), and the 43805692nd base (allele G) in chromosome 2 of GRCH37/hg19 THADA (THADA armadillo repeat containing); or
A second mutation that reduces the function of the THADA protein and has an allele frequency of 5% or less.
2. The composition of item 1, wherein the second mutation is a missense mutation, frameshift mutation, nonsense mutation, or splice site mutation.
3. The composition of item 1, wherein the agent is a primer or probe that specifically binds to the THADA gene.
4. The composition of item 1, wherein the second mutation is selected from the group consisting of: mutations of the 43230964th base (allele C), 43231141st base (allele A), 43231295th base (allele T), 43231306th base (allele A), 43231306th base (allele C), 43231323rd base (allele T), 43232797th base (allele G), 43232820th base (allele A), 43232850th base (allele T), 43232874th base (allele C), 43232880th base (mutation from ACT to allele A), 43279821st base (allele A), 43279824th base (allele C), 43279840th base (allele T), 43279855th base (allele C), 43279867th base (allele G), 43279875th base (allele G), 43286968th base (allele T), 43286970th base (allele C), 43286995th base (allele A), 43286998th base (allele T), 43287058th base (allele C), 43291749th base (allele A), 43292125th base (allele G), 43292130th base (allele T), 43292144th base (allele T), 43292201st base (allele C), 43292983rd base (allele A), 43293195th base (allele G), 43320475th base (allele G), 43344122nd base (allele G), 43344138th base (allele A), 43397971st base (allele G), 43397976th base (allele C), 43398003rd base (allele A), 43398005th base (allele T), 43398030th base (allele A), 43398105th base (allele C), 43428128th base (allele C), 43428130th base (allele A), 43428131st base (allele A), 43428134th base (allele C), 43428194th base (allele C), 43430242nd base (allele A), 43430259th base (allele A), 43430261st base (allele C), 43430265th base (allele T), 43430277th base (allele A), 43430288th base (allele C), 43485283rd base (allele C), 43485286th base (allele C), 43485321st base (allele T), 43485322nd base (allele A), 43498843rd base (allele A), 43498867th base (allele A), 43498868th base (allele A), 43498876th base (allele A), 43498877th base (allele G), 43498912th base (allele T), 43498913th base (allele A), 43498915th base (allele A), 43498930th base (allele A), 43498937th base (allele A), 43498941st base (mutation from AT to allele A), 43505722nd base (allele A), 43508647th base (allele A), 43508653rd base (allele C), 43508657th base (allele C), 43508676th base (allele T), 43508677th base (allele C), 43508683rd base (allele C), 43508688th base (allele T), 43508710th base (allele C), 43508751st base (allele A), 43520160th base (allele C), 43527891st base (allele C), 43527894th base (allele T), 43527898th base (allele A), 43527934th base (allele T), 43527966th base (allele G), 43527970th base (allele C), 43541167th base (allele T), 43541237th base (allele A), 43541254th base (allele C), 43541296th base (allele T), 43541298th base (mutation from CAT to allele C), 43549266th base (allele C), 43549356th base (allele T), 43549363rd base (allele T), 43551812th base (allele G), 43551815th base (allele T), 43551906th base (allele T), 43552202nd base (allele T), 43552203rd base (allele G), 43552203rd base (allele G), 43552246th base (allele T), 43552255th base (allele G), 43552256th base (allele C), 43552279th base (allele G), 43552285th base (allele A), 43552311th base (allele G), 43552312th base (allele G), 43552316th base (allele A), 43556356th base (allele C), 43556467th base (allele G), 43556467th base (allele T), 43556495th base (allele A), 43556522nd base (allele T), 43560299th base (allele A), 43560334th base (allele C), 43560359th base (allele A), 43566712th base (allele C), 43566745th base (allele A), 43566760th base (allele A), 43566766th base (allele T), 43566785th base (allele T), 43566791st base (allele G), 43566808th base (allele A), 43570390th base (allele C), 43570392nd base (allele C), 43570471st base (allele G), 43570490th base (allele G), 43571736th base (allele A), 43571736th base (mutation from G to allele GCA), 43571742nd base (allele T), 43571757th base (allele C), 43571759th base (allele T), 43571760th base (allele C), 43571762nd base (allele C), 43571766th base (allele G), 43571775th base (allele G), 43571804th base (allele A), 43571822nd base (allele T), 43571858th base (allele T), 43572900th base (allele T), 43572911th base (allele A), 43572949th base (allele G), 43574347th base (allele A), 43574378th base (allele C), 43574429th base (allele C), 43574432nd base (allele T), 43574440th base (allele A), 43574473rd base (allele T), 43574474th base (allele C), 43574477th base (allele A), 43574551st base (allele G), 43574606th base (allele G), 43574690th base (allele G), 43574693rd base (allele A), 43574714th base (allele A), 43574729th base (allele A), 43574745th base (allele G), 43574800th base (allele C), 43574824th base (allele G), 43574838th base (allele T), 43574849th base (allele T), 43574860th base (allele C), 43574861st base (allele A), 43574875th base (allele C), 43574878th base (allele G), 43574881st base (allele C), 43574882nd base (allele G), 43574901st base (allele C), 43574929th base (allele A), 43574962nd base (allele G), 43575013th base (allele A), 43575019th base (allele C), 43575025th base (mutation from AT to allele A), 43577064th base (allele C), 43577122nd base (mutation from CCT to allele C), 43577151st base (allele C), 43577166th base (allele T), 43578565th base (allele G), 43578577th base (allele T), 43578583rd base (allele T), 43581834th base (allele A), 43586411th base (allele T), 43586884th base (allele C), 43586893rd base (allele C), 43586897th base (allele G), 43586909th base (allele C), 43586941st base (allele C), 43586965th base (allele G), 43590824th base (allele G), 43590888th base (allele G), 43590947th base (allele C), 43590951st base (mutation from CAATCTAT to allele C), 43591954th base (allele C), 43591986th base (allele A), 43591998th base (allele A), 43592040th base (allele T), 43592328th base (allele G), 43592335th base (allele T), 43592390th base (allele T), 43625187th base (allele C), 43625278th base (allele A) and 43818077th base (allele C) in chromosome 2 of GRCH37/hg19.
5. A method for providing information to predict sulfonylurea dependency in diabetes mellitus patients, comprising identifying at least one of the following:
A first mutation selected from the group consisting of mutations of the 43801878th base (allele A), the 43801909th base (allele T), the 43776463rd base (allele C), the 43625187th base (allele C), the 43520160th base (allele C), and the 43805692nd base (allele G) in chromosome 2 of GRCH37/hg19 THADA (THADA armadillo repeat containing); or
A second mutation that reduces the function of the THADA protein and has an allele frequency of 5% or less.
6. The method of item 5, wherein the second mutation is a missense mutation, frameshift mutation, nonsense mutation, or splice site mutation.
7. The method of item 5, wherein the agent is a primer or probe that specifically binds to the THADA gene.
8. The method of item 5, wherein the second mutation is selected from the group consisting of: mutations of the 43230964th base (allele C), 43231141st base (allele A), 43231295th base (allele T), 43231306th base (allele A), 43231306th base (allele C), 43231323rd base (allele T), 43232797th base (allele G), 43232820th base (allele A), 43232850th base (allele T), 43232874th base (allele C), 43232880th base (mutation from ACT to allele A), 43279821st base (allele A), 43279824th base (allele C), 43279840th base (allele T), 43279855th base (allele C), 43279867th base (allele G), 43279875th base (allele G), 43286968th base (allele T), 43286970th base (allele C), 43286995th base (allele A), 43286998th base (allele T), 43287058th base (allele C), 43291749th base (allele A), 43292125th base (allele G), 43292130th base (allele T), 43292144th base (allele T), 43292201st base (allele C), 43292983rd base (allele A), 43293195th base (allele G), 43320475th base (allele G), 43344122nd base (allele G), 43344138th base (allele A), 43397971st base (allele G), 43397976th base (allele C), 43398003rd base (allele A), 43398005th base (allele T), 43398030th base (allele A), 43398105th base (allele C), 43428128th base (allele C), 43428130th base (allele A), 43428131st base (allele A), 43428134th base (allele C), 43428194th base (allele C), 43430242nd base (allele A), 43430259th base (allele A), 43430261st base (allele C), 43430265th base (allele T), 43430277th base (allele A), 43430288th base (allele C), 43485283rd base (allele C), 43485286th base (allele C), 43485321st base (allele T), 43485322nd base (allele A), 43498843rd base (allele A), 43498867th base (allele A), 43498868th base (allele A), 43498876th base (allele A), 43498877th base (allele G), 43498912th base (allele T), 43498913th base (allele A), 43498915th base (allele A), 43498930th base (allele A), 43498937th base (allele A), 43498941st base (mutation from AT to allele A), 43505722nd base (allele A), 43508647th base (allele A), 43508653rd base (allele C), 43508657th base (allele C), 43508676th base (allele T), 43508677th base (allele C), 43508683rd base (allele C), 43508688th base (allele T), 43508710th base (allele C), 43508751st base (allele A), 43520160th base (allele C), 43527891st base (allele C), 43527894th base (allele T), 43527898th base (allele A), 43527934th base (allele T), 43527966th base (allele G), 43527970th base (allele C), 43541167th base (allele T), 43541237th base (allele A), 43541254th base (allele C), 43541296th base (allele T), 43541298th base (mutation from CAT to allele C), 43549266th base (allele C), 43549356th base (allele T), 43549363rd base (allele T), 43551812th base (allele G), 43551815th base (allele T), 43551906th base (allele T), 43552202nd base (allele T), 43552203rd base (allele G), 43552203rd base (allele G), 43552246th base (allele T), 43552255th base (allele G), 43552256th base (allele C), 43552279th base (allele G), 43552285th base (allele A), 43552311th base (allele G), 43552312th base (allele G), 43552316th base (allele A), 43556356th base (allele C), 43556467th base (allele G), 43556467th base (allele T), 43556495th base (allele A), 43556522nd base (allele T), 43560299th base (allele A), 43560334th base (allele C), 43560359th base (allele A), 43566712th base (allele C), 43566745th base (allele A), 43566760th base (allele A), 43566766th base (allele T), 43566785th base (allele T), 43566791st base (allele G), 43566808th base (allele A), 43570390th base (allele C), 43570392nd base (allele C), 43570471st base (allele G), 43570490th base (allele G), 43571736th base (allele A), 43571736th base (mutation from G to allele GCA), 43571742nd base (allele T), 43571757th base (allele C), 43571759th base (allele T), 43571760th base (allele C), 43571762nd base (allele C), 43571766th base (allele G), 43571775th base (allele G), 43571804th base (allele A), 43571822nd base (allele T), 43571858th base (allele T), 43572900th base (allele T), 43572911th base (allele A), 43572949th base (allele G), 43574347th base (allele A), 43574378th base (allele C), 43574429th base (allele C), 43574432nd base (allele T), 43574440th base (allele A), 43574473rd base (allele T), 43574474th base (allele C), 43574477th base (allele A), 43574551st base (allele G), 43574606th base (allele G), 43574690th base (allele G), 43574693rd base (allele A), 43574714th base (allele A), 43574729th base (allele A), 43574745th base (allele G), 43574800th base (allele C), 43574824th base (allele G), 43574838th base (allele T), 43574849th base (allele T), 43574860th base (allele C), 43574861st base (allele A), 43574875th base (allele C), 43574878th base (allele G), 43574881st base (allele C), 43574882nd base (allele G), 43574901st base (allele C), 43574929th base (allele A), 43574962nd base (allele G), 43575013th base (allele A), 43575019th base (allele C), 43575025th base (mutation from AT to allele A), 43577064th base (allele C), 43577122nd base (mutation from CCT to allele C), 43577151st base (allele C), 43577166th base (allele T), 43578565th base (allele G), 43578577th base (allele T), 43578583rd base (allele T), 43581834th base (allele A), 43586411th base (allele T), 43586884th base (allele C), 43586893rd base (allele C), 43586897th base (allele G), 43586909th base (allele C), 43586941st base (allele C), 43586965th base (allele G), 43590824th base (allele G), 43590888th base (allele G), 43590947th base (allele C), 43590951st base (mutation from CAATCTAT to allele C), 43591954th base (allele C), 43591986th base (allele A), 43591998th base (allele A), 43592040th base (allele T), 43592328th base (allele G), 43592335th base (allele T), 43592390th base (allele T), 43625187th base (allele C), 43625278th base (allele A) and 43818077th base (allele C) in chromosome 2 of GRCH37/hg19.
9. The method of item 5, further comprising providing information that an individual possessing the first or second mutation exhibits sulfonylurea dependency.

### Effect of the Invention

The present invention enables the prediction of whether a diabetes mellitus patient exhibits excellent blood glucose-lowering effects with sulfonylureas using THADA (THADA armadillo repeat containing) as a marker, and provides information for this purpose.

### BRIEF DESCRIPTION OF THE DRAWINGS

FIG. 1 illustrates a schematic method for deriving sulfonylurea dependency-related variants according to the present invention. DM, diabetes mellitus; FPG, fasting plasma glucose; MAF, minor allele frequency; SNV, single nucleotide variant; SU, sulfonylurea; WES, whole exome sequencing.

FIG. 2 shows the blood levels of HbA1c (glycated hemoglobin) or FPG (fasting plasma glucose) after discontinuation of sulfonylurea administration in a retrospective study. Values are expressed as mean±standard deviation. ***, p<0.001 between the two groups by two-way repeated measures ANOVA.

FIG. 3 illustrates a schematic method for prospectively verifying THADA candidate variants according to the present invention. FPG, fasting plasma glucose; SU, sulfonylurea; WES, whole exome sequencing.

FIG. 4 shows the blood levels of HbA1c (glycated hemoglobin) or FPG (fasting plasma glucose) after discontinuation of sulfonylurea administration in a prospective study. Values are expressed as mean±standard deviation. **, p<0.01 between the two groups by two-way ANOVA.

FIG. 5 illustrates a schematic method for verifying THADA variants in a large population cohort using UK Biobank registrants. eGFR, estimated glomerular filtration rate calculated by the CKD-EPI method; SU, sulfonylureas.

FIG. 6 illustrates a schematic method for verifying THADA variants in a large population cohort using publicly available genetic databases. SU, sulfonylureas; WES, whole exome sequencing. dbNSFP v4 (http://database.liulab.science/dbNSFP). gnomAD exomes v2.1.1 (https://gnomad.broadinstitute.org).

### FORM FOR PRACTICING THE INVENTION

The present invention will now be described in detail. Unless specifically defined otherwise, all terms in this specification shall have the same general meaning as understood by a person skilled in the technical field to which the present invention belongs. In the case of any conflict between the meaning of a term used in this specification and its general meaning, the meaning used in this specification shall prevail.

The present invention relates to a composition for predicting sulfonylurea dependence in diabetic patients, which comprises a reagent to identify at least one first mutation selected from the group consisting of mutations in the 43801878th base (allele A), the 43801909th base (allele T), the 43776463rd base (allele C), the 43625187th base (allele C), the 43520160th base (allele C), and the 43805692nd base (allele G) of chromosome 2 in GRCh37/hg19 of THADA (THADA armadillo repeat containing); or a second mutation that impairs the function of THADA protein and has an allele frequency of 5% or less.

In the present invention, the term “sulfonylurea dependence” refers to the degree to which a diabetic patient requires sulfonylurea exclusively or preferentially compared to other medications for blood glucose control. That is, it means that blood glucose reduction is not observed or the effect is not as expected when taking non-sulfonylurea conventional hypoglycemic agents, such as metformin, gliptin, or pioglitazone, but the predicted glucose-lowering effect can only be observed when sulfonylurea is taken, thereby necessitating the preferential use of sulfonylurea.

In the present invention, the composition for predicting sulfonylurea dependence comprises a reagent for detecting at least one first mutation selected from the group consisting of mutations in the 43801878th base (allele A), the 43801909th base (allele T), the 43776463rd base (allele C), the 43625187th base (allele C), the 43520160th base (allele C), and the 43805692nd base (allele G) of chromosome 2 in GRCh37/hg19 of THADA (THADA armadillo repeat containing); or a second mutation that impairs the function of THADA protein and has an allele frequency of 5% or less, thereby predicting whether a diabetic patient has sulfonylurea dependence. Specifically, the possession of the first or second mutation can predict high dependence on sulfonylurea. However, this is not limiting.

The second mutation impairs the function of THADA protein and has an allele frequency of 5% or less. Such mutations are not limited by type, location, or altered base, as long as they impair the protein function. Mutations that impair protein function may, for example, be those predicted to change function using known methods such as SIFT or PolyPhen. Since many mutations in THADA are known, including their mutation frequency, impact by type, and predicted protein function, it is easy to determine whether a mutation qualifies as a second mutation using resources such as dbNSFP and gnomAD.

In the present invention, the mutations (first or second mutations) of the gene are included in the scope of the invention without limitation, as long as the base sequence of the gene has been added, deleted, or altered. For example, they may be missense mutations, frameshift mutations, nonsense mutations, or splice site mutations. Preferably, they may cause mutations affecting the protein function of the gene in the present invention. However, this is not limiting.

The second mutation may include mutations such as those at the 43230964th base (allele C), 43231141st base (allele A), 43231295th base (allele T), 43231306th base (allele A), 43231306th base (allele C), 43231323rd base (allele T), 43232797th base (allele G), 43232820th base (allele A), 43232850th base (allele T), 43232874th base (allele C), 43232880th base (allele A with ACT mutated to A), 43279821st base (allele A), 43279824th base (allele C), 43279840th base (allele T), 43279855th base (allele C), 43279867th base (allele G), 43279875th base (allele G), 43286968th base (allele T), 43286970th base (allele C), 43286995th base (allele A), 43286998th base (allele T), 43287058th base (allele C), 43291749th base (allele A), 43292125th base (allele G), 43292130th base (allele T), 43292144th base (allele T), 43292201st base (allele C), 43292983rd base (allele A), 43293195th base (allele G), 43320475th base (allele G), 43344122nd base (allele G), 43344138th base (allele A), 43397971st base (allele G), 43397976th base (allele C), 43398003rd base (allele A), 43398005th base (allele T), 43398030th base (allele A), 43398105th base (allele C), 43428128th base (allele C), 43428130th base (allele A), 43428131st base (allele A), 43428134th base (allele C), 43428194th base (allele C), 43430242nd base (allele A), 43430259th base (allele A), 43430261st base (allele C), 43430265th base (allele T), 43430277th base (allele A), 43430288th base (allele C), 43485283rd base (allele C), 43485286th base (allele C), 43485321st base (allele T), 43485322nd base (allele A), 43498843rd base (allele A), 43498867th base (allele A), 43498868th base (allele A), 43498876th base (allele A), 43498877th base (allele G), 43498912th base (allele T), 43498913th base (allele A), 43498915th base (allele A), 43498930th base (allele A), 43498937th base (allele A), 43498941st base (allele A with AT mutated to A), 43505722nd base (allele A), 43508647th base (allele A), 43508653rd base (allele C), 43508657th base (allele C), 43508676th base (allele T), 43508677th base (allele C), 43508683rd base (allele C), 43508688th base (allele T), 43508710th base (allele C), 43508751st base (allele A), 43520160th base (allele C), 43527891st base (allele C), 43527894th base (allele T), 43527898th base (allele A), 43527934th base (allele T), 43527966th base (allele G), 43527970th base (allele C), 43541167th base (allele T), 43541237th base (allele A), 43541254th base (allele C), 43541296th base (allele T), 43541298th base (allele CAT mutated to allele C), 43549266th base (allele C), 43549356th base (allele T), 43549363rd base (allele T), 43551812th base (allele G), 43551815th base (allele T), 43551906th base (allele T), 43552202nd base (allele T), 43552203rd base (allele G), 43552203rd base (allele G), 43552246th base (allele T), 43552255th base (allele G), 43552256th base (allele C), 43552279th base (allele G), 43552285th base (allele A), 43552311th base (allele G), 43552312th base (allele G), 43552316th base (allele A), 43556356th base (allele C), 43556467th base (allele G), 43556467th base (allele T), 43556495th base (allele A), 43556522nd base (allele T), 43560299th base (allele A), 43560334th base (allele C), 43560359th base (allele A), 43566712th base (allele C), 43566745th base (allele A), 43566760th base (allele A), 43566766th base (allele T), 43566785th base (allele T), 43566791st base (allele G), 43566808th base (allele A), 43570390th base (allele C), 43570392nd base (allele C), 43570471st base (allele G), 43570490th base (allele G), 43571736th base (allele A), 43571736th base (allele G mutated to GCA), 43571742nd base (allele T), 43571757th base (allele C), 43571759th base (allele T), 43571760th base (allele C), 43571762nd base (allele C), 43571766th base (allele G), 43571775th base (allele G), 43571804th base (allele A), 43571822nd base (allele T), 43571858th base (allele T), 43572900th base (allele T), 43572911th base (allele A), 43572949th base (allele G), 43574347th base (allele A), 43574378th base (allele C), 43574429th base (allele C), 43574432nd base (allele T), 43574440th base (allele A), 43574473rd base (allele T), 43574474th base (allele C), 43574477th base (allele A), 43574551st base (allele G), 43574606th base (allele G), 43574690th base (allele G), 43574693rd base (allele A), 43574714th base (allele A), 43574729th base (allele A), 43574745th base (allele G), 43574800th base (allele C), 43574824th base (allele G), 43574838th base (allele T), 43574849th base (allele T), 43574860th base (allele C), 43574861st base (allele A), 43574875th base (allele C), 43574878th base (allele G), 43574881st base (allele C), 43574882nd base (allele G), 43574901st base (allele C), 43574929th base (allele A), 43574962nd base (allele G), 43575013th base (allele A), 43575019th base (allele C), 43575025th base (allele AT mutated to A), 43577064th base (allele C), 43577122nd base (allele CCT mutated to C), 43577151st base (allele C), 43577166th base (allele T), 43578565th base (allele G), 43578577th base (allele T), 43578583rd base (allele T), 43581834th base (allele A), 43586411th base (allele T), 43586884th base (allele C), 43586893rd base (allele C), 43586897th base (allele G), 43586909th base (allele C), 43586941st base (allele C), 43586965th base (allele G), 43590824th base (allele G), 43590888th base (allele G), 43590947th base (allele C), 43590951st base (allele CAATCTAT mutated to C), 43591954th base (allele C), 43591986th base (allele A), 43591998th base (allele A), 43592040th base (allele T), 43592328th base (allele G), 43592335th base (allele T), 43592390th base (allele T), 43625187th base (allele C), 43625278th base (allele A), 43818077th base (allele C). However, this is not limiting.

THADA genes may have the above-mentioned sequences. The sequences for each species are all publicly known. For example, in humans, the gene may correspond to Gene ID: 63892.

In the present invention, the reagent for identifying the above mutations (first or second mutations) may be used without limitation as long as it can confirm the sequence, presence of the mutation, etc. For example, the reagent may be a primer, probe, or antisense nucleic acid, but is not limited thereto. Using the primer, probe, or antisense nucleic acid, the nucleic acid sequence with a specific allele at the mutation site can be amplified or detected.

In the present invention, diabetic patients are included within the scope of the invention regardless of the type of diabetes. Preferably, it may be Type 2 Diabetes Mellitus (T2DM), but this is not limiting.

The present invention also relates to a method for providing information for predicting sulfonylurea dependence in diabetic patients, which includes identifying at least one first mutation selected from the group consisting of mutations in the 43801878th base (allele A), 43801909th base (allele T), 43776463rd base (allele C), 43625187th base (allele C), 43520160th base (allele C), and the 43805692nd base (allele G) of chromosome 2 in GRCh37/hg19 of THADA (THADA armadillo repeat containing); or at least one second mutation that impairs the function of THADA protein and has an allele frequency of 5% or less in a biological sample isolated from a subject.

The second mutation may include the aforementioned mutations.

The method for providing information for predicting sulfonylurea dependence in diabetic patients may further include the step of amplifying and sequencing DNA from a biological sample isolated from a subject to obtain the subject's genotype information. The methods for DNA amplification, sequencing analysis, and genotype information acquisition can be used without limitation as long as they are known to those skilled in the art.

Furthermore, the method for providing information in the present invention may include the step of providing information indicating that an individual with the first or second mutation has sulfonylurea dependence.

Hereinafter, the present invention will be described in detail with examples to concretely explain the invention.

### Experimental Example 1. Identification of SU-Dependent Patients Through Retrospective Medical Record Review

A retrospective observational study was conducted on adult patients with type 2 diabetes mellitus (T2DM) who visited Seoul National University Hospital between 2009 and 2015. These patients had been taking low-dose sulfonylureas (SU, glimepiride equivalent≤2 mg/day) and discontinued their use.

SU dependency was defined as meeting all the following criteria:
(1) Patients with HbA1c≤7.0% or HbA1c≤7.5% (in the case of hypoglycemic risk) for at least six months while taking low-dose SU (glimepiride equivalent≤2 mg/day).
(2) Patients who showed an increase in HbA1c>1.2% within three months or an increase in HbA1c>1.5% within six months after discontinuing SU, regardless of the replacement of SU with other oral diabetes medications.
(3) Patients who resumed SU administration.
(4) Patients who exhibited a decrease in HbA1c>0.8% or a reduction in fasting blood glucose>40 mg/dl within three months of resuming SU.

Patients were considered SU-independent if their HbA1c remained below 7.5% after discontinuing SU, and SU was not resumed until the final data collection in 2019. The SU-independent control group was selected from T2DM patients with a disease duration of over 10 years and was matched with SU-dependent patients in terms of age, sex, and anti-diabetic medications.

Patients were excluded from analysis if they had impaired renal function (serum creatinine>1.4 mg/dl or estimated glomerular filtration rate [eGFR]<50 ml/min/1.73 m²), clinically significant liver disease, were taking medications such as glucocorticoids that could affect glycemic control, or experienced serious medical issues during changes in SU use.

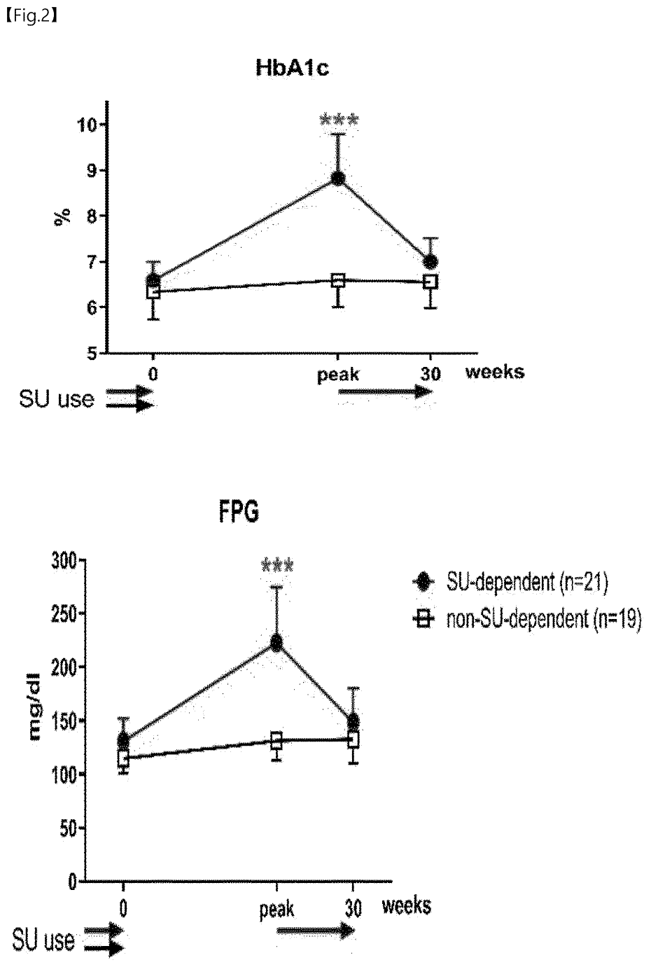

As a result, 21 SU-dependent patients and 19 SU-independent control patients were selected. SU-dependent patients showed significant increases in blood glucose levels after SU discontinuation (HbA1c increased from 6.6±0.4% to 8.8±1.0% at 20 weeks) and significant reductions in blood glucose after resuming SU (HbA1c decreased to 6.9±0.5% at 12 weeks), as well as similar trends in fasting blood glucose. This indicated a significantly distinct drug response compared to the SU-independent control group (FIG. 2). However, clinical characteristics and anti-diabetic medication use before SU discontinuation were similar in both groups (Table 1), suggesting difficulty in distinguishing SU responsiveness prior to discontinuation.

Comparisons of Clinical Characteristics and Anti-Diabetic Medications Before And after SU Discontinuation

TABLE 1

| | | SU- | Non-SU- | |
|---|---|---|---|---|
| | | dependent | dependent | |
| | | (n = 21) | (n = 19) | P* |
| Male (%) | | 62 | 58 | 1.000 |
| Age (years) | | 66.1 ± 9.9 | 66.3 ± 7.5 | 0.966 |
| Duration of diabetes (years) | | 16.0 (5~36) | 16.7 ± 3.9 | 0.291 |
| Body mass index (kg/m²) | | 24.7 ± 3.0 | 25.5 ± 4.6 | 0.524 |
| T2DM in the 1^{st}-degree | | 81 | 75 | 0.705 |
| family (%) | | | | |
| Fasting C-peptide (nmol/L) | | 0.99 ± 0.57 | 0.94 ± 0.44 | 0.873 |
| | | (n = 11) | (n = 8) | |
| Dyslipidemia^{†} (%) | | 90 | 95 | 1.000 |
| Hypertension (%) | | 90 | 89 | 1.000 |
| Ischemic heart diseases (%) | | 19 | 11 | 0.381 |
| Ischemic stroke (%) | | 19 | 11 | 0.381 |
| Urine albumin/creatinine | | 12 (4~127) | 23 (0~97) | 0.154 |
| ratio (mg/g) | | | | |
| eGFR (mL/min/1.73 m²) | | 80.5 ± 18.7 | 82.2 ± 18.0 | 0.775 |
| Retinopathy (%) | | 48 | 26 | 0.144 |
| SU dose (mg/day as | | 0.90 ± 0.36 | 0.95 ± 1.05 | 0.832 |
| glimepiride-equivalent [2,3]) | | | | |
| Anti-diabetic | Metformin | 86 | 95 | 0.607 |
| agents before SU | Gliptins | 38 | 32 | 0.748 |
| discontinuation (%) | Pioglit- | 19 | 21 | 1.000 |
| | azone | | | |
| Anti-diabetic | Metformin | 90 | 100 | 1.000 |
| agents during SU | Gliptins | 62 | 68 | 0.748 |
| discontinuation (%) | Pioglit- | 29 | 21 | 0.721 |
| | azone | | | |
| Anti-diabetic | Metformin | 90 | Not | |
| agents after SU | Gliptins | 43 | applicable | |
| resumption (%) | Pioglit- | 19 | | |
| | azone | | | |

Data are expressed as mean±standard deviation or median (range).

eGFR: estimated glomerular filtration rate; SU: sulfonylureas; T2DM: type 2 diabetes mellitus.

*Analyzed by Student's t-test, Mann-Whitney test, or Fisher's exact test.

### Experimental Example 2. Whole-Exome Sequencing (WES)

To analyze the genetic characteristics of SU-dependent patients, whole-exome sequencing was planned to identify rare variants with large effect sizes due to the limited sample size. Blood samples for DNA extraction were collected from 17 of the 21 SU-dependent patients who voluntarily submitted written consent.

The study was conducted in accordance with the Declaration of Helsinki and Good Clinical Practice principles, and was approved by the Institutional Review Board (#1407-103-596). Exome sequencing was performed by Macrogen (Seoul, Korea). Briefly, DNA was extracted from blood leukocytes, exome capturing was performed using SureSelect v4+UTR (Agilent Technologies, Santa Clara, CA, USA), and sequencing was carried out on a HiSeq 2000 sequencing system (Illumina Inc., San Diego, CA, USA) with 100-fold coverage. Sequence reads were aligned to the UCSC genome assembly hg19 using BWA, and variants were called and annotated using SAMtools and ANNOVAR software.

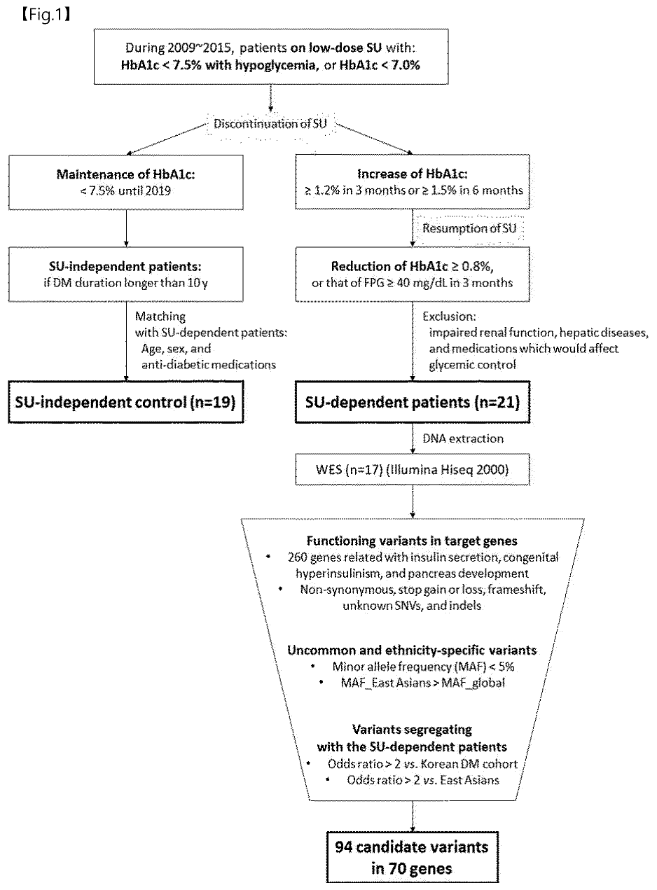

### Experimental Example 3. Selection of Candidate Variants (FIG. 1)

To conduct gene-based analysis, 260 target genes associated with beta-cell function and mass were selected. Single nucleotide variants (SNVs) altering amino acid sequences were screened from these genes based on the whole-exome sequencing results. Since type 2 diabetes is a complex disease influenced significantly by rare variants (Am J Hum Genet. 2001; 69:124), and considering the small sample size of this study, the analysis was limited to uncommon variants (minor allele frequency<5%, 1000 Genomes Phase 1).

It is known that variations in drug-response genes exhibit significant differences among ethnic groups (Genome Med. 2017; 9 (1): 117). The high usage of SU among East Asians (EAS) suggests good SU response in this population (Diabetes Obes Metab. 2023 January; 25 (1): 208). Therefore, variants with higher minor allele frequencies in East Asians (EAS) compared to the global average frequency were selected (Genome Aggregation Database [gnomAD], v2.1.1).

To further select variants enriched in SU-dependent patients, variants with odds ratios≥2 were identified by comparison with two control groups: either East Asians (gnomAD_EAS) or the Korean T2DM cohort (SNUH Project, http://koex.snu.ac.kr). As a result, 94 single nucleotide variants (SNVs) in 70 genes were selected as candidate SU dependency-related variants (Table 2).

TABLE 2
Rare/Amino acid-changing/East Asian-enriched/SU-
dependent patient-focused SNV candidates

| | |
|---|---|
| 1 | ABCC8:NM_000352:exon12:c.G1678A:p.V560M, |
| 2 | ACLY:NM_198830:exon18:c.C2093T:p.P698L, |
| | ACLY:NM_001096:exon19:c.C2123T:p.P708L, |
| 3 | AKAP5:NM_004857:exon2:c.G871A:p.G291R, |
| 4 | APOA5:NM_052968:exon4:c.G538C:p.V180L, |
| | APOA5:NM_001166598:exon4:c.G538C:p.V180L, |
| 5 | APOA5:NM_052968:exon4:c.G553T:p.G185C, |
| | APOA5:NM_001166598:exon4:c.G553T:p.G185C, |
| 6 | ARAP1:NM_001040118:exon18:c.G2518A:p.E840K, |
| | ARAP1:NM_015242:exon16:c.G1783A:p.E595K, |
| | ARAP1:NM_001135190:exon15:c.G1600A:p.E534K, |
| 7 | ARAP1:NM_001040118:exon7:c.G883A:p.G295R, |
| | ARAP1:NM_015242:exon5:c.G148A:p.G50R, |
| | ARAP1:NM_001135190:exon5:c.G148A:p.G50R, |
| 8 | BLK:NM_001715:exon4:c.G252C:p.K84N, |
| 9 | CACNA1A:NM_001127222:exon19:c.C2759G:p.A920G, |
| | CACNA1A:NM_001127221:exon19:c.C2762G:p.A921G, |
| 10 | CACNA1D:NM_001128839:exon30:c.C3854T:p.A1285V, |
| | CACNA1D:NM_001128840:exon30:c.C3854T:p.A1285V, |
| | CACNA1D:NM_000720:exon31:c.C3914T:p.A1305V, |
| 11 | CACNA1E:NM_001205293:exon20:c.C27131T:p.R911W, |
| | CACNA1E:NM_000721:exon20:c.C2731T:p.R911W, |
| | CACNA1E:NM_001205294:exon19:c.C2674T:p.R892W, |
| 12 | CACNA1H:NM_021098:exon10:c.C2030T:p.S677L, |
| | CACNA1H:NM_001005407:exon10:c.C2030T:p.S677L, |
| 13 | CAPN10:NM_023083:exon4:c.C598A:p.P200T, |
| | CAPN10:NM_023085:exon4:c.C598A:p.P200T, |
| 14 | CASR:NM_000388:exon4:c.G1192A:p.D398N, |
| | CASR:NM_001178065:exon4:c.G1192A:p.D398N, |
| 15 | CASR:NM_000388:exon7:c.G2824A:p.E942K, |
| | CASR:NM_001178065:exon7:c.G2854A:p.E952K, |
| 16 | CCKAR:NM_000730:exon2:c.A305G:p.N102S, |
| 17 | CD38:NM_001775:exon3:c.C418T:p.R140W, |
| 18 | DIS3L2:NM_152383:exon13:c.G1448A:p.R483Q, |
| 19 | DIS3L2:NM_152383:exon13:c.G1600A:p.G534R, |
| 20 | EIF2AK3:NM_004836:exon12:c.G2014A:p.E672K, |
| 21 | EPHA5:NM_004439:exon17:c.A2876G:p.H959R, |
| | EPHA5:NM_182472:exon16:c.A2810G:p.H937R, |
| 22 | FAM3D:NM_138805:exon5:c.C178T:p.P60S, |
| 23 | FASN:NM_004104:exon22:c.G3557T:p.C1186F, |
| 24 | FASN:NM_004104:exon24:c.C4127T:p.A1376V, |
| 25 | FASN:NM_004104:exon34:c.G5809A:p.V1937I, |
| 26 | FASN:NM_004104:exon42:c.C7301T:p.T2434I, |
| 27 | FASN:NM_004104:exon42:c.G7192T:p.A2398S, |
| 28 | FOXA2:NM_021784:exon2:c.G274A:p.A92T, |
| | FOXA2:NM_153675:exon3:c.G256A:p.A86T, |
| 29 | GAL:NM_015973:exon6:c.C368T:p.S123F, |
| 30 | GHSR:NM_198407:exon2:c.G1070A:p.R357Q, |
| 31 | GIPR:NM_000164:exon11:c.G947T:p.R316L, |
| 32 | GIPR:NM_000164:exon6:c.C406T:p.R136W, |
| 33 | GIPR:NM_000164:exon9:c.C843A:p.Y281X, |
| 34 | GLIS3:NM_152629:exon3:c.G951T:p.E317D, |
| | GLIS3:NM_001042413:exon4:c.G1416T:p.E472D, |
| 35 | GLIS3:NM_152629:exon7:c.G1819A:p.A607T, |
| | GLIS3:NM_001042413:exon8:c.G2284A:p.A762T, |
| 36 | GLIS3:NM_152629:exon8:c.A2006G:p.H669R, |
| | GLIS3:NM_001042413:exon9:c.A2471G:p.H824R, |
| 37 | GLP1R:NM_002062:exon2:c.G131A:p.R44H, |
| 38 | GPLD1:NM_001503:exon21:c.T2081C:p.M694T, |
| 39 | HLA-DRB5:NM_002125:exon2:c.C165A:p.F55L, |
| 40 | HNF4A:NM_001030003:exon2:c.C83T:p.A28V, |
| | HNF4A:NM_178850:exon2:c.C149T:p.A50V, |
| | HNF4A:NM_178849:exon2:c.C149T:p.A50V, |
| | HNF4A:NM_000457:exon2:c.C149T:p.A50V, |
| | HNF4A:NM_175914:exon2:c.C83T:p.A28V, |
| | HNF4A:NM_001030004:exon2:c.C83T:p.A28V, |
| 41 | HTT:NM_002111:exon12:c.G1652A:p.G551E, |
| 42 | INHBB:NM_002193:exon1:c.G157A:p.D53N, |
| 43 | IRS1:NM_005544:exon1:c.G2164A:p.G722S, |
| 44 | KANK1:NM_015158:exon3:c.C1991T:p.A664V, |
| | KANK1:NM_153186:exon2:c.C1517T:p.A506V, |
| 45 | KANK1:NM_015158:exon3:c.G1196A:p.R399Q, |
| | KANK1:NM_153186:exon2:c.G722A:p.R241Q, |
| 46 | KANK1:NM_015158:exon3:c.G1915A:p.V639I, |
| | KANK1:NM_153186:exon2:c.G1441A:p.V481I, |
| 47 | KCNB2:NM_004770:exon3:c.C2279A:p.T760N, |
| 48 | KCNH6:NM_030779:exon6:c.G1280A:p.R427Q, |
| 49 | KCNH6:NM_030779:exon6:c.G1354A:p.D452N, |
| 50 | KCNJ15:NM_170736:exon3:c.A88C:p.M30L, |
| | KCNJ15:NM_002243:exon4:c.A88C:p.M30L, |
| | KCNJ15:NM_170737:exon3:c.A88C:p.M30L, |
| 51 | KCNQ1:NM_000218:exon10:c.C1343G:p.P448R, |
| 52 | LBR:NM_002296:exon12:c.G1528A:p.A510T, |
| | LBR:NM_194442:exon12:c.G1528A:p.A510T, |
| 53 | LRP5:NM_002335:exon2:c.C290T:p.A97V, |
| 54 | LRP5:NM_002335:exon9:c.G1871A:p.R624Q, |
| 55 | MARCKS:NM_002356:exon2:c.C821T:p.A274V, |
| 56 | MC4R:NM_005912:exon1:c.G914A:p.R305Q, |
| 57 | MGEA5:NM_012215:exon1:c.G137A:p.G46E, |
| | MGEA5:NM_001142434:exon1:c.G137A:p.G46E, |
| 58 | MICU1:NM_001195519:exon3:c.T215G:p.L72R, |
| | MICU1:NM_001195518:exon8:c.T809G:p.L270R, |
| 59 | MYO5A:NM_000259:exon20:c.C2491T:p.R831C, |
| | MYO5A:NM_001142495:exon20:c.C2491T:p.R831C, |
| 60 | NNT:NM_182977:exon14:c.C1987T:p.L663F, |
| | NNT:NM_012343:exon14:c.C1987T:p.L663F, |
| 61 | NNT:NM_182977:exon3:c.A188G:p.K63R, |
| | NNT:NM_012343:exon3:c.A188G:p.K63R, |
| 62 | NOS2:NM_000625:exon19:c.A2239G:p.T747A, |
| 63 | NR0B2:NM_021969:exon1:c.C100T:p.R34X, |
| 64 | OXCT1:NM_000436:exon2:c.C173T:p.T58M, |
| 65 | PAM:NM_138822:exon19:c.T2153C:p.F718S, |
| | PAM:NM_001177306:exon19:c.T2153C:p.F718S, |
| | PAM:NM_000919:exon19:c.T2153C:p.F718S, |
| | PAM:NM_138766:exon19:c.T2153C:p.F718S, |
| | PAM:NM_138821:exon18:c.T1832C:p.F611S, |
| 66 | PAX4:NM_006193:exon1:c.G92A:p.R31Q, |
| 67 | PC:NM_001040716:exon15:c.A1702G:p.T568A, |
| | PC:NM_000920:exon14:c.A1702G:p.T568A, |
| | PC:NM_022172:exon13:c.A1702G:p.T568A, |
| 68 | PCK2:NM_004563:exon9:c.C1379T:p.P460L, |
| 69 | PDE4C:NM_001098818:exon1:c.C88T:p.L30F, |
| 70 | PDE8B:NM_001029853:exon18:c.C2084T:p.T695M, |
| | PDE8B:NM_003719:exon19:c.C2144T:p.T715M, |
| | PDE8B:NM_001029852:exon18:c.C1979T:p.T660M, |
| | PDE8B:NM_001029851:exon16:c.C1853T:p.T618M, |
| | PDE8B:NM_001029854:exon18:c.C2003T:p.T668M, |
| 71 | PRKCE:NM_005400:exon9:c.G1130C:p.R377P, |
| 72 | PSMD9:NM_002813:exon1:c.G31A:p.G11S, |
| 73 | RAPGEF3:NM_001098531:exon2:c.C79T:p.R27W, |
| 74 | REST:NM_001193508:exon4:c.A2241G:p.I747M, |
| | REST:NM_005612:exon4:c.A2241G:p.I747M, |
| 75 | RYR2:NM_001035:exon46:c.G7076A:p.R2359Q, |
| 76 | RYR2:NM_001035:exon56:c.A8419G:p.I2807V, |
| 77 | RYR3:NM_001243996:exon39:c.A6125C:p.N2042T, |
| | RYR3:NM_001036:exon39:c.A6125C:p.N2042T, |
| 78 | SCN9A:NM_002977:exon15:c.A2359G:p.M787V, |
| 79 | SIRT4:NM_012240:exon3:c.G703A:p.G235R, |
| 80 | SLC18A2:NM_003054:exon10:c.C899G:p.S300C, |
| 81 | SLC24A6:NM_024959:exon11:c.C1072T:p.L358F, |
| 82 | SLC2A2:NM_000340:exon3:c.G301A:p.V101I, |
| 83 | THADA:NM_022065:exon11:c.G1326T:p.E442D, |
| | THADA:NM_001083953:exon11:c.G1326T:p.E442D, |
| 84 | THADA:NM_022065:exon11:c.T1295A:p.V432D, |
| | THADA:NM_001083953:exon11:c.T1295A:p.V432D, |
| 85 | THADA:NM_022065:exon20:c.C2992G:p.R998G, |
| | THADA:NM_001083953:exon20:c.C2992G:p.R998G, |
| 86 | THADA:NM_022065:exon29:c.C4150G:p.R1384G, |
| | THADA:NM_001083953:exon29:c.C4150G:p.R1384G, |
| 87 | THADA:NM_022065:exon32:c.C4631G:p.S1544C, |
| | THADA:NM_001083953:exon32:c.C4631G:p.S1544C, |
| 88 | THADA:NM_022065:exon9:c.T776C:p.I259T, |
| | THADA:NM_001083953:exon9:c.T776C:p.I259T, |
| 89 | TRPM2:NM_003307:exon11:c.A1748G:p.N583S, |
| 90 | TRPM2:NM_003307:exon26:c.C3830T:p.T1277M, |
| 91 | TRPM4:NM_017636:exon4:c.C290T:p.T97M, |
| | TRPM4:NM_001195227:exon4:c.C290T:p.T97M, |
| 92 | TRPM5:NM_014555:exon5:c.G655C:p.G219R, |
| 93 | TRPV3:NM_145068:exon5:c.G443A:p.R148Q, |
| 94 | WFS1:NM_001145853:exon2:c.A41G:p.Q14R, |
| | WFS1:NM_006005:exon2:c.A41G:p.Q14R, |
| — | — |
| — | — |
| — | — |
| — | — |
| — | — |
| — | — |

### Example 1. Validation of THADA Candidate Mutations in Prospectively Identified SU-Dependent Patients

Among the 70 selected genes, most contained only one or two candidate mutations, whereas THADA exhibited six candidate mutations observed in five of 17 patients (Serial Nos. 83-88). To verify whether these candidate mutations in THADA are associated with SU responsiveness, a clinical study was conducted to prospectively identify patients dependent on SU and perform genetic testing. (IRB No. H 1906 070 1041)

In Experimental Example 1, approximately 20% of enrolled patients were calculated to exhibit SU dependency. To select 10 SU-dependent patients, 50 patients were required, and accounting for a 15% dropout rate, a total of 58 patients were planned for enrollment. The inclusion criteria were as follows: patients with type 2 diabetes mellitus who had been taking a low dose of SU (equivalent to ≤2 mg/day of glimepiride) for more than six months, maintaining an HbA1c of ≤7.5%. After obtaining written informed consent from the patients, SU was discontinued and replaced with other oral antidiabetic agents (DPP-4 inhibitors or thiazolidinediones). To determine SU dependency, blood glucose and HbA1c levels were observed for up to 20 weeks. If blood glucose significantly worsened (defined as any of the following: an increase in HbA1c of ≥1% within 12 weeks, an increase in HbA1c of ≥1.5% within 20 weeks, or an increase in fasting blood glucose of >80 mg/dl within 20 weeks), the patient was determined to be SU-dependent, and SU treatment was resumed.

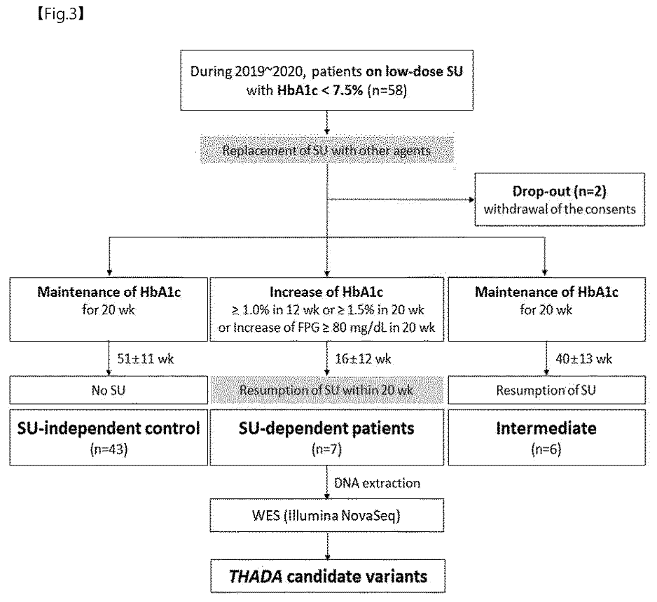

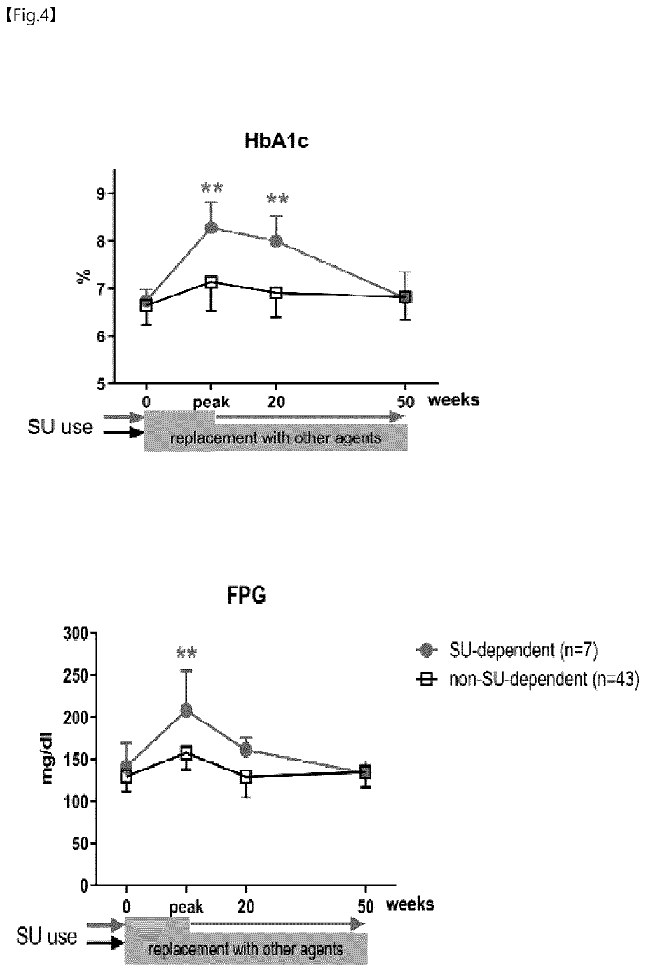

As a result of the study, two patients withdrew their consent and were excluded, seven patients were found to be SU-dependent, and 43 patients were not. An additional six patients did not resume SU during the 20-week observation period but later resumed SU (average SU discontinuation period of 40 weeks), making their dependency unclear (FIG. 3). The clinical characteristics of prospectively identified SU-dependent patients showed no statistically significant differences compared to non-SU-dependent patients, consistent with findings from the retrospective study (Tables 1 and 3). Their patterns of blood glucose changes are presented in FIG. 4.

### Clinical Characteristics of Prospectively Verified SU-Dependent Patients

TABLE 3

| | SU- | Non-SU- | |
|---|---|---|---|
| | dependent | dependent | |
| | (n = 7) | (n = 43) | P* |
| Male (%) | 6 (86) | 28 (65) | 0.279 |
| Age (years) | 61.6 ± 7.1 | 67.3 ± 8.3 | 0.095 |
| Weight (kg) | 63.5 ± 11.0 | 67.8 ± 9.9 | 0.297 |
| Body mass index (kg/m²) | 23.6 ± 3.2 | 25.7 ± 3.0 | 0.097 |
| Duration of diabetes (years) | 18.1 ± 7.3 | 18.1 ± 4.1 | 0.840 |
| T2DM in the 1^{st}- | 1 (14) | 18 (42) | 0.229 |
| degree family (9%) | | | |
| Systolic blood | 129.1 ± 12.7 | 136.3 ± 15.1 | 0.245 |
| pressure (mmHg) | | | |
| Diastolic blood | 73.7 ± 8.4 | 79.1 ± 10.1 | 0.185 |
| pressure (mmHg) | | | |
| Glycated hemoglobin (%) | 6.7 ± 0.2 | 6.6 ± 0.4 | 0.535 |
| Fasting plasma | 136.3 ± 30.3 | 135.2 ± 24.5 | 0.916 |
| glucose (mg/dL) | | | |
| Fasting C-peptide (nmol/L) | 0.52 ± 0.11 | 0.67 ± 0.23 | 0.117 |
| Total cholesterol (mg/dL) | 145.4 ± 24.5 | 139.3 ± 23.3 | 0.524 |
| Triglyceride (mg/dL) | 86.3 ± 34.4 | 119.9 ± 98.2 | 0.377 |
| Urine albumin/creatinine | 26.3 ± 15.8 | 34.2 ± 47.5 | 0.691 |
| ratio (mg/g) | | | |
| eGFR (mL/min/1.73 m²) | 77.27 ± 16.45 | 83.15 ± 12.78 | 0.283 |
| Duration of SU use (year) | 12.3 ± 5.4 | 8.9 ± 6.6 | 0.209 |
| SU, n (%) | | | |
| Glimepiride | 4 (57) | 25 (58) | |
| Gliclazide | 3 (43) | 18 (42) | 1.000 |

Exome sequencing (performed by Macrogen, Korea; see Experimental Example 2) was conducted on seven prospectively confirmed SU-dependent patients to analyze whether the THADA candidate mutations (Table 2, Serial Nos. 83-88) were observed. Mutations 85, 86, and 88 were identified in two patients. Considering that these mutations are rare (approximately 0.1%) in East Asians (Table 4), the repeated observation of these mutations in these small number of SU-dependent patients in both retrospective (FIG. 1) and prospective (FIG. 3) studies suggests a significant association between these mutations and SU dependency.

In summary, when candidate mutations in THADA were examined in seven patients who restarted SU treatment due to worsening blood glucose within 20 weeks after switching from low-dose SU to other antidiabetic agents, three out of six candidate mutations were observed. Given the rarity of these mutations, it can be interpreted that they are associated with SU dependency.

THADA Mutations Associated with SU Dependency

TABLE 4

| Gene | | | | | | | | | | |
|---|---|---|---|---|---|---|---|---|---|---|
| Entry | | | | | | | | | | |
| number | | Position | | | MAF | MAF | MAF | Korean | UKBB | |
| in table 2 | | (GRCh37/ | | | Global | EAS | EUR | Example 1 | Example 2 | |
| THADA | rsiD | hg19) | Reference | Alternate | (%) | (%) | (%) | (Person) | (Person) | Effect^{a} |
| 83 | rs182434450 | 2-43801878 | C | A | 0.006 | 0.089 | ND | 0 | NA | N |
| 84 | rs763574266 | 2-43801909 | A | T | 0.004 | 0.061 | ND | 0 | 0 | N |
| 85 | rs143275203 | 2-43776463 | G | C | 0.005 | 0.071 | ND | 1 | 0 | P |
| 86 | rs548266644 | 2-43625187 | G | C | 0.010 | 0.145 | ND | 1 | 1 | N |
| 87 | rs57257569 | 2-43520160 | G | C | 0.108 | 1.33 | 0.003 | 0 | 8 | P |
| 88 | rs779034878 | 2-43805692 | A | G | 0.002 | 0.022 | ND | 1 | NA | N |

| | | | | | | | |
|---|---|---|---|---|---|---|---|
| NA | 197 variants from open sources^{b} | <0.68 | NA | NA | NA | 1,335 | P |

a, frameshift, stop-gained, splice donor, splice acceptor, or functional prediction by either Polyphen or SIFT
b, dbNSFP v4. (http://database.liulab.science/dbNSFP) and genomAD exomes v2.1.1 (https://gnomad.broadinstitute.org)
EAS; East Asian; EUR, European; MAF, minor allele frequency (genomAD exomes v2.1.1); N, not predicted by either Polyphen or SIFT; NA, not applicable; ND, not detected; P, predicted function; UKBB, UK Biobank

### Example 2. Validation of SU Drug Effects According to THADA Variations in a Large Population Group

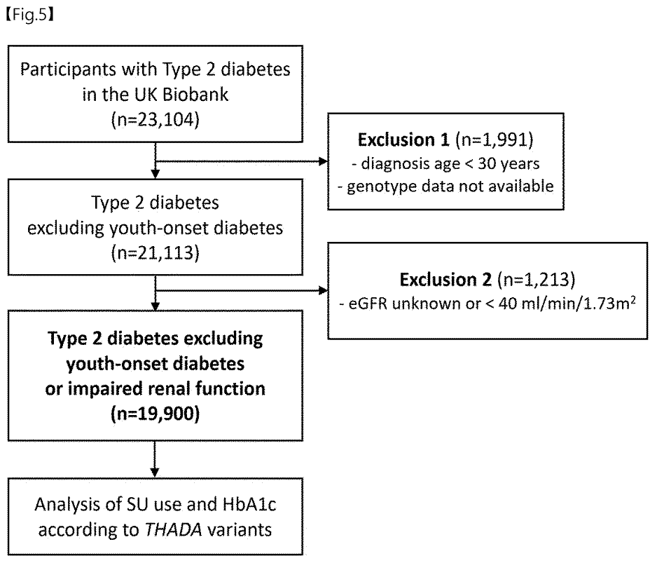

To analyze whether candidate variations in THADA are associated with SU response in a large population group, the UK Biobank data was utilized. The UK Biobank is a community-based cohort in the UK, with approximately 500,000 adult participants registered between 2006 and 2010 (Nucleic acids research 47.D1 (2019): D1005-D1012). Among them, 23,104 participants were coded as having type 2 diabetes. To exclude cases of type 1 diabetes or atypical youth-onset diabetes, participants with an age of onset below 30 years were excluded. Additionally, 1,991 participants without genetic information were also excluded. Furthermore, 1,213 participants with significant kidney dysfunction (GFR<40 ml/min/1.73 m2), which could significantly affect glycemic control and prescription of anti-diabetics, were excluded. Consequently, 19,900 participants were included in the final analysis (FIG. 5).

Since the candidate variations identified in Experimental Example 3 were specific to East Asians, only 9 out of 19,900 participants in the predominantly European Caucasian UK Biobank carried the six THADA candidate variants (Table 4). Considering the low frequency of candidate variations and the small cohort of Korean patients, it was hypothesized that some candidate THADA variations could have been missed in the Experimental Example 3. Therefore, additional THADA SNVs (single nucleotide variations) predicted to affect protein function were screened using the publicly available SNV database, dbNSFP (Genome Medicine. (2020) 12:103). The screening criteria included variations with a frequency below 5% and those predicted to alter protein function based on in silico predictions using Polyphen or SIFT. Furthermore, predicted loss-of-function (pLoF) variations were identified in the gnomAD v2.1.1 browser, including SNVs and multi-nucleotide variants causing the following changes: transcript_ablation, splice_acceptor_variant, splice_donor_variant, stop_gained, and frameshift_variant.

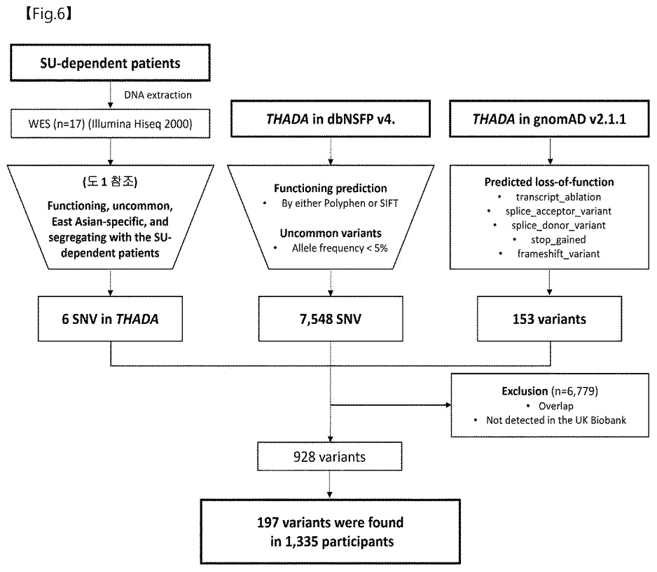

From the two databases, a total of 7,548 variations from the former and 153 variations from the latter were identified. Among these, 928 variations, including the six candidate variations from Korean SU-dependent patients, were extracted from the UK Biobank, and 197 of these variations were found in 1,335 participants (FIG. 6, Table 7).

Subsequently, the 1,335 variant carriers (test group) were compared to the remaining 18,565 participants (control group) (Table 5). The test group exhibited significantly younger ages and earlier ages of diagnosis compared to the control group. Even after propensity score matching (PSM, 1:10) adjustment for age, sex, and BMI, the test group still showed a younger age of diagnosis, indicating that variation carriers are at higher risk of developing diabetes.

### Clinical Characteristics According to the Presence of Variations Identified in THADA

TABLE 5

| | | Mutation | P- |
|---|---|---|---|
| Variable | Control Group | carrier group | value |
| Number of Subjects | 18,565 | 1,335 | |
| Age | 60.24 ± 6.78 | 59.57 ± 7.05 | <0.001 |
| Male Sex, % | 63.25 | 60.67 | 0.063 |
| BMI (Kg/m²) | 31.62 ± 5.76 | 31.70 ± 6.13 | 0.644 |
| Diagnosis Age | 55.09 ± 7.64 | 54.10 ± 7.99 | <0.001 |
| Number of Subjects | 13,250 | 1,325 | |
| (PSM) | | | |
| Age (PSM matched | 59.64 ± 7.05 | 59.61 ± 7.02 | 0.892 |
| variable) | | | |
| Male Sex, % (PSM | 60.42 | 60.6 | 0.917 |
| matched variable) | | | |
| BMI (Kg/m²) (PSM | 31.69 ± 5.80 | 31.70 ± 6.13 | 0.986 |
| matched variable) | | | |
| Diagnosis Age (PSM | 54.59 ± 7.83 | 54.13 ± 7.99 | 0.048 |
| compared variable) | | | |

BMI, body mass index; PSM, propensity score matching for age, sex, and BMI

### Mean±SD

At the time of registration in the UK Biobank, the typical sequence of drug usage in Europe was metformin, sulfonylureas (SU), and insulin. In the early stages of diagnosis, it is difficult to attribute drug usage patterns to genetic influences. However, as the disease duration increases, prescription patterns begin to reflect blood glucose responses to medications. In other words, over time, feedback from drug efficacy influences prescriptions, aligning them more closely with genetic susceptibility. Therefore, if variant carriers exhibit favorable blood glucose responses to SU, the proportion of SU prescriptions would increase with longer disease duration, leading to improved glycemic control and a reduced proportion of insulin use.

To analyze this, participants were divided based on a diabetes duration threshold of 72 months. The drug prescription patterns and glycemic control of the test group and the control group were then compared (Table 6).

### Clinical Characteristics According to the Presence of THADA Variations by Diabetes Duration

TABLE 6

| | Diabetes Duration < 72 | | | Diabetes Duration ≥ 72 | | |
|---|---|---|---|---|---|---|
| | Months (Median: 30 Months) | | | Months (Median: 114 Months) | | |
| | Control | Mutation | P- | Control | Mutation | P- |
| Variable | Group | Carrier | value | Group | Carrier | value |
| Number of | 12,200 | 864 | | 6,365 | 471 | |
| Subjects | | | | | | |
| Age | 59.59 ± | 59.09 ± | 0.0496 | 61.49 ± | 60.43 ± | 0.0011 |
| | 6.96 | 7.14 | | 6.25 | 6.8 | |
| Male Sex, % | 61.61 | 58.56 | 0.0812 | 66.39 | 64.54 | 0.4417 |
| BMI | 31.63 ± | 31.77 ± | 0.5303 | 31.59 ± | 31.57 ± | 0.9528 |
| (Kg/m²) | 5.75 | 6.09 | | 5.77 | 6.22 | |
| Diabetes | 28.85 ± | 30.17 ± | 0.0932 | 124.82 ± | 130.78 ± | 0.0217 |
| Duration | 21.43 | 22.26 | | 52.41 | 54.31 | |
| (Months) | | | | | | |
| HbA1c | 50.6 ± | 51.88 ± | 0.0166 | 54.76 ± | 54.73 ± | 0.9719 |
| (mmol/mol) | 13.21 | 14.75 | | 13.92 | 14.23 | |
| MTF User, % | 52.03 | 56.83 | 0.0071 | 68.08 | 70.28 | 0.348 |
| SU User, % | 15.63 | 16.09 | 0.7576 | 40.22 | 45.65 | 0.0235 |
| Insulin | 8.84 | 7.52 | 0.2058 | 15.15 | 12.95 | 0.2224 |
| User, % | | | | | | |
| Number of | 8,570 | 857 | | 4,680 | 468 | |
| Subjects | | | | | | |
| (PSM) | | | | | | |
| Age (PSM | 59.21 ± | 59.16 ± | 0.8292 | 60.48 ± | 60.44 ± | 0.8984 |
| matched | 7.09 | 7.1 | | 6.58 | 6.82 | |
| variable) | | | | | | |
| Male Sex, % | 58.73 | 58.58 | 0.9604 | 64.57 | 64.32 | 0.9522 |
| (PSM | | | | | | |
| matched | | | | | | |
| variable) | | | | | | |
| BMI (Kg/m²) | 31.75 ± | 31.77 ± | 0.9342 | 31.53 ± | 31.57 ± | 0.8997 |
| (PSM | 5.82 | 6.09 | | 5.81 | 6.22 | |
| matched | | | | | | |
| variable) | | | | | | |
| Diabetes | 30.12 ± | 30.26 ± | 0.9128 | 130.22 ± | 130.87 ± | 0.8057 |
| Duration | 21.36 | 22.28 | | 56.69 | 54.4 | |
| (Months) | | | | | | |
| (PSM | | | | | | |
| matched | | | | | | |
| variable) | | | | | | |
| eGFR | 92.85 ± | 92.83 ± | 0.9595 | 91.06 ± | 91.21 ± | 0.845 |
| (ml/min/ | 15.19 | 15.14 | | 16.52 | 15.98 | |
| 1.73 m²) | | | | | | |
| (PSM | | | | | | |
| matched | | | | | | |
| variable) | | | | | | |
| HbA1c | 50.75 ± | 51.86 ± | 0.0406 | 55.01 ± | 54.73 ± | 0.6954 |
| (mmol/mol) | 13.34 | 14.78 | | 14.28 | 14.28 | |
| (PSM | | | | | | |
| compared | | | | | | |
| variable) | | | | | | |
| MTF User, % | 52.73 | 56.71 | 0.0286 | 68.65 | 70.3 | 0.4964 |
| (PSM | | | | | | |
| compared | | | | | | |
| variable) | | | | | | |
| SU User, % | 16.34 | 15.99 | 0.829 | 41.35 | 45.73 | 0.0746 |
| (PSM | | | | | | |
| compared | | | | | | |
| variable) | | | | | | |
| Insulin | 9.39 | 7.58 | 0.0925 | 15.11 | 13.03 | 0.2577 |
| User, % | | | | | | |
| (PSM | | | | | | |
| compared | | | | | | |
| variable) | | | | | | |

BMI, body mass index; eGFR, estimated glomerular filtration rate; MTF, metformin; SU, sulfonylurea; PSM, propensity score matching for age, sex, BMI, diabetes duration, and eGFR

### Mean±SD

When the disease duration was short, the glycated hemoglobin (HbA1c) levels and the proportion of metformin users were significantly higher in variant carriers. This remained true even after adjustments for age, sex, BMI, disease duration, and renal function (eGFR) using propensity score matching (1:10). This suggests that variant carriers, identified as high-risk for diabetes development (Table 5), exhibited poorer glycemic control, leading to the earlier prescription of the first-line medication, metformin.

For participants with a disease duration of 72 months or more (median: 114 months) compared to those with a shorter duration (median: 30 months), the SU usage rate increased more than twofold (approximately 15% vs. 40%). Among these individuals, there was no significant difference between the test group and the control group in terms of HbA1c levels or metformin usage. However, the proportion of SU users was significantly higher in the test group. After adjustments for age, sex, BMI, disease duration, and renal function using propensity score matching (1:10), the statistical significance disappeared, but the SU usage rate remained higher (41.3% Vs. 45.7%, P=0.0746). This suggests that when SU was added as a second-line medication for prolonged diabetes, its usage increased more in variant carriers, thereby reducing the glycemic differences between them and the control group that were observed in patients with earlier-stage diabetes.

Notably, the proportion of insulin prescriptions, typically initiated upon failure of SU treatment, tended to be lower in variant carriers regardless of disease duration (Table 6). After combining all disease durations and adjusting for age, sex, BMI, disease duration, and renal function, the insulin prescription rate was significantly different: 9.51% in the test group and 11.43% in the control group (P=0.0387). This indicates that variant carriers responded well to SU, achieving better glycemic control and delaying insulin prescription.

To summarize Example 2, an analysis of UK Biobank data revealed that THADA variant carriers are high-risk diabetes patients with poor glycemic control and higher early usage of the first-line medication, metformin. However, with longer disease durations, as the proportion of SU users increased, their glycemic control became similar to that of the control group. Furthermore, their lower rate of insulin use suggests that THADA variant carriers respond well to SU.

In conclusion, multiple candidate variations in THADA were identified retrospectively from SU-dependent patients (FIG. 1, FIG. 2, Table 2), and three of them were prospectively validated in seven SU-dependent patients, even though the variants are reported very rare: around 0.1% in East Asians (Example 1: FIG. 3, FIG. 4, Table 4). Additionally, by including additional rare functional variations predicted to alter THADA protein function, an analysis of UK Biobank data demonstrated that, as disease duration lengthens and genetic susceptibility shapes the prescription patterns, THADA variant carriers experience improved glycemic control and reduced insulin use, with increased SU usage (Example 2: FIG. 5, FIG. 6, Table 6, Table 7).

Although pharmacogenetics in type 2 diabetes mellitus (T2DM) is not currently applied in clinical practice, it has potential for application in subgroups such as SU-dependent patients. This study identified novel nucleotide variants associated with SU responsiveness in T2DM, which could contribute to predicting SU response through genetic analysis.

TABLE 7

| Chromosome position | | |
|---|---|---|
| and allele change | | |
| (GRCh37) | Reference | Alteration |
| 2.43230964.G.C | G | C |
| 2.43231141.G.A | G | A |
| 2.43231295.C.T | C | T |
| 2.43231306.T.A | T | A |
| 2.43231306.T.C | T | C |
| 2.43231323.A.T | A | T |
| 2.43232797.C.G | C | G |
| 2.43232820.C.A | C | A |
| 2.43232850.C.T | C | T |
| 2.43232874.A.C | A | C |
| 2.43232880.ACT.A | ACT | A |
| 2.43279821.G.A | G | A |
| 2.43279824.T.C | T | C |
| 2.43279840.C.T | C | T |
| 2.43279855.G.C | G | C |
| 2.43279867.A.G | A | G |
| 2.43279875.A.G | A | G |
| 2.43286968.C.T | C | T |
| 2.43286970.G.C | G | C |
| 2.43286995.G.A | G | A |
| 2.43286998.C.T | C | T |
| 2.43287058.T.C | T | C |
| 2.43291749.C.A | C | A |
| 2.43292125.A.G | A | G |
| 2.43292130.C.T | C | T |
| 2.43292144.C.T | C | T |
| 2.43292201.T.C | T | C |
| 2.43292983.G.A | G | A |
| 2.43293195.A.G | A | G |
| 2.43320475.A.G | A | G |
| 2.43344122.C.G | C | G |
| 2.43344138.G.A | G | A |
| 2.43397971.C.G | C | G |
| 2.43397976.G.C | G | C |
| 2.43398003.G.A | G | A |
| 2.43398005.A.T | A | T |
| 2.43398030.G.A | G | A |
| 2.43398105.T.C | T | C |
| 2.43428128.T.C | T | C |
| 2.43428130.C.A | C | A |
| 2.43428131.T.A | T | A |
| 2.43428134.G.C | G | C |
| 2.43428194.G.C | G | C |
| 2.43430242.C.A | C | A |
| 2.43430259.G.A | G | A |
| 2.43430261.T.C | T | C |
| 2.43430265.G.T | G | T |
| 2.43430277.G.A | G | A |
| 2.43430288.T.C | T | C |
| 2.43485283.T.C | T | C |
| 2.43485286.T.C | T | C |
| 2.43485321.C.T | C | T |
| 2.43485322.G.A | G | A |
| 2.43498843.G.A | G | A |
| 2.43498867.G.A | G | A |
| 2.43498868.C.A | C | A |
| 2.43498876.C.A | C | A |
| 2.43498877.C.G | C | G |
| 2.43498912.C.T | C | T |
| 2.43498913.G.A | G | A |
| 2.43498915.G.A | G | A |
| 2.43498930.G.A | G | A |
| 2.43498937.G.A | G | A |
| 2.43498941.AT.A | AT | A |
| 2.43505722.G.A | G | A |
| 2.43508647.C.A | C | A |
| 2.43508653.T.C | T | C |
| 2.43508657.G.C | G | C |
| 2.43508676.C.T | C | T |
| 2.43508677.G.C | G | C |
| 2.43508683.T.C | T | C |
| 2.43508688.C.T | C | T |
| 2.43508710.T.C | T | C |
| 2.43508751.G.A | G | A |
| 2.43520160.G.C | G | C |
| 2.43527891.T.C | T | C |
| 2.43527894.G.T | G | T |
| 2.43527898.G.A | G | A |
| 2.43527934.C.T | C | T |
| 2.43527966.A.G | A | G |
| 2.43527970.A.C | A | C |
| 2.43541167.C.T | C | T |
| 2.43541237.T.A | T | A |
| 2.43541254.T.C | T | C |
| 2.43541296.C.T | C | T |
| 2.43541298.CAT.C | CAT | C |
| 2.43549266.T.C | T | C |
| 2.43549356.C.T | C | T |
| 2.43549363.C.T | C | T |
| 2.43551812.A.G | A | G |
| 2.43551815.C.T | C | T |
| 2.43551906.C.T | C | T |
| 2.43552202.A.T | A | T |
| 2.43552203.C.G | C | G |
| 2.43552203.C.G | C | G |
| 2.43552246.C.T | C | T |
| 2.43552255.A.G | A | G |
| 2.43552256.T.C | T | C |
| 2.43552279.A.G | A | G |
| 2.43552285.G.A | G | A |
| 2.43552311.C.G | C | G |
| 2.43552312.T.G | T | G |
| 2.43552316.G.A | G | A |
| 2.43556356.T.C | T | C |
| 2.43556467.A.G | A | G |
| 2.43556467.A.T | A | T |
| 2.43556495.G.A | G | A |
| 2.43556522.C.T | C | T |
| 2.43560299.C.A | C | A |
| 2.43560334.A.C | A | C |
| 2.43560359.T.A | T | A |
| 2.43566712.A.C | A | C |
| 2.43566745.G.A | G | A |
| 2.43566760.G.A | G | A |
| 2.43566766.G.T | G | T |
| 2.43566785.C.T | C | T |
| 2.43566791.A.G | A | G |
| 2.43566808.G.A | G | A |
| 2.43570390.T.C | T | C |
| 2.43570392.T.C | T | C |
| 2.43570471.T.G | T | G |
| 2.43570490.T.G | T | G |
| 2.43571736.G.A | G | A |
| 2.43571736.G.GCA | G | GCA |
| 2.43571742.C.T | C | T |
| 2.43571757.T.C | T | C |
| 2.43571759.A.T | A | T |
| 2.43571760.G.C | G | C |
| 2.43571762.T.C | T | C |
| 2.43571766.A.G | A | G |
| 2.43571775.A.G | A | G |
| 2.43571804.G.A | G | A |
| 2.43571822.C.T | C | T |
| 2.43571858.C.T | C | T |
| 2.43572900.C.T | C | T |
| 2.43572911.G.A | G | A |
| 2.43572949.C.G | C | G |
| 2.43574347.T.A | T | A |
| 2.43574378.A.C | A | C |
| 2.43574429.T.C | T | C |
| 2.43574432.C.T | C | T |
| 2.43574440.T.A | T | A |
| 2.43574473.A.T | A | T |
| 2.43574474.G.C | G | C |
| 2.43574477.G.A | G | A |
| 2.43574551.T.G | T | G |
| 2.43574606.C.G | C | G |
| 2.43574690.C.G | C | G |
| 2.43574693.G.A | G | A |
| 2.43574714.T.A | T | A |
| 2.43574729.G.A | G | A |
| 2.43574745.C.G | C | G |
| 2.43574800.T.C | T | C |
| 2.43574824.A.G | A | G |
| 2.43574838.G.T | G | T |
| 2.43574849.C.T | C | T |
| 2.43574860.T.C | T | C |
| 2.43574861.G.A | G | A |
| 2.43574875.T.C | T | C |
| 2.43574878.A.G | A | G |
| 2.43574881.T.C | T | C |
| 2.43574882.A.G | A | G |
| 2.43574901.T.C | T | C |
| 2.43574929.G.A | G | A |
| 2.43574962.A.G | A | G |
| 2.43575013.G.A | G | A |
| 2.43575019.T.C | T | C |
| 2.43575025.AT.A | AT | A |
| 2.43577064.A.C | A | C |
| 2.43577122.CCT.C | CCT | C |
| 2.43577151.G.C | G | C |
| 2.43577166.C.T | C | T |
| 2.43578565.A.G | A | G |
| 2.43578577.G.T | G | T |
| 2.43578583.C.T | C | T |
| 2.43581834.C.A | C | A |
| 2.43586411.C.T | C | T |
| 2.43586884.T.C | T | C |
| 2.43586893.T.C | T | C |
| 2.43586897.T.G | T | G |
| 2.43586909.G.C | G | C |
| 2.43586941.G.C | G | C |
| 2.43586965.C.G | C | G |
| 2.43590824.C.G | C | G |
| 2.43590888.C.G | C | G |
| 2.43590947.G.C | G | C |
| 2.43590951.CAATCTAT.C | CAATCTAT | C |
| 2.43591954.G.C | G | C |
| 2.43591986.G.A | G | A |
| 2.43591998.C.A | C | A |
| 2.43592040.G.T | G | T |
| 2.43592328.T.G | T | G |
| 2.43592335.C.T | C | T |
| 2.43592390.C.T | C | T |
| 2.43625187.G.C | G | C |
| 2.43625278.C.A | C | A |
| 2.43818077.T.C | T | C |

(Only these 3 are based on GRCh37/hg19. The preceding 194 are based on GRCh38.)

1. A composition for predicting sulfonylurea dependency in diabetes mellitus patients, comprising an agent that identifies at least one of the following:
A first mutation selected from the group consisting of mutations of the 43801878th base (allele A), the 43801909th base (allele T), the 43776463rd base (allele C), the 43625187th base (allele C), the 43520160th base (allele C), and the 43805692nd base (allele G) in chromosome 2 of GRCH37/hg19 THADA (THADA armadillo repeat containing); or
A second mutation that reduces the function of the THADA protein and has an allele frequency of 5% or less.

2. The composition of claim 1, wherein the second mutation is a missense mutation, frameshift mutation, nonsense mutation, or splice site mutation.

3. The composition of claim 1, wherein the agent is a primer or probe that specifically binds to the THADA gene.

4. The composition of claim 1, wherein the second mutation is selected from the group consisting of: mutations of the 43230964th base (allele C), 43231141st base (allele A), 43231295th base (allele T), 43231306th base (allele A), 43231306th base (allele C), 43231323rd base (allele T), 43232797th base (allele G), 43232820th base (allele A), 43232850th base (allele T), 43232874th base (allele C), 43232880th base (mutation from ACT to allele A), 43279821st base (allele A), 43279824th base (allele C), 43279840th base (allele T), 43279855th base (allele C), 43279867th base (allele G), 43279875th base (allele G), 43286968th base (allele T), 43286970th base (allele C), 43286995th base (allele A), 43286998th base (allele T), 43287058th base (allele C), 43291749th base (allele A), 43292125th base (allele G), 43292130th base (allele T), 43292144th base (allele T), 43292201st base (allele C), 43292983rd base (allele A), 43293195th base (allele G), 43320475th base (allele G), 43344122nd base (allele G), 43344138th base (allele A), 43397971st base (allele G), 43397976th base (allele C), 43398003rd base (allele A), 43398005th base (allele T), 43398030th base (allele A), 43398105th base (allele C), 43428128th base (allele C), 43428130th base (allele A), 43428131st base (allele A), 43428134th base (allele C), 43428194th base (allele C), 43430242nd base (allele A), 43430259th base (allele A), 43430261st base (allele C), 43430265th base (allele T), 43430277th base (allele A), 43430288th base (allele C), 43485283rd base (allele C), 43485286th base (allele C), 43485321st base (allele T), 43485322nd base (allele A), 43498843rd base (allele A), 43498867th base (allele A), 43498868th base (allele A), 43498876th base (allele A), 43498877th base (allele G), 43498912th base (allele T), 43498913th base (allele A), 43498915th base (allele A), 43498930th base (allele A), 43498937th base (allele A), 43498941st base (mutation from AT to allele A), 43505722nd base (allele A), 43508647th base (allele A), 43508653rd base (allele C), 43508657th base (allele C), 43508676th base (allele T), 43508677th base (allele C), 43508683rd base (allele C), 43508688th base (allele T), 43508710th base (allele C), 43508751st base (allele A), 43520160th base (allele C), 43527891st base (allele C), 43527894th base (allele T), 43527898th base (allele A), 43527934th base (allele T), 43527966th base (allele G), 43527970th base (allele C), 43541167th base (allele T), 43541237th base (allele A), 43541254th base (allele C), 43541296th base (allele T), 43541298th base (mutation from CAT to allele C), 43549266th base (allele C), 43549356th base (allele T), 43549363rd base (allele T), 43551812th base (allele G), 43551815th base (allele T), 43551906th base (allele T), 43552202nd base (allele T), 43552203rd base (allele G), 43552203rd base (allele G), 43552246th base (allele T), 43552255th base (allele G), 43552256th base (allele C), 43552279th base (allele G), 43552285th base (allele A), 43552311th base (allele G), 43552312th base (allele G), 43552316th base (allele A), 43556356th base (allele C), 43556467th base (allele G), 43556467th base (allele T), 43556495th base (allele A), 43556522nd base (allele T), 43560299th base (allele A), 43560334th base (allele C), 43560359th base (allele A), 43566712th base (allele C), 43566745th base (allele A), 43566760th base (allele A), 43566766th base (allele T), 43566785th base (allele T), 43566791st base (allele G), 43566808th base (allele A), 43570390th base (allele C), 43570392nd base (allele C), 43570471st base (allele G), 43570490th base (allele G), 43571736th base (allele A), 43571736th base (mutation from G to allele GCA), 43571742nd base (allele T), 43571757th base (allele C), 43571759th base (allele T), 43571760th base (allele C), 43571762nd base (allele C), 43571766th base (allele G), 43571775th base (allele G), 43571804th base (allele A), 43571822nd base (allele T), 43571858th base (allele T), 43572900th base (allele T), 43572911th base (allele A), 43572949th base (allele G), 43574347th base (allele A), 43574378th base (allele C), 43574429th base (allele C), 43574432nd base (allele T), 43574440th base (allele A), 43574473rd base (allele T), 43574474th base (allele C), 43574477th base (allele A), 43574551st base (allele G), 43574606th base (allele G), 43574690th base (allele G), 43574693rd base (allele A), 43574714th base (allele A), 43574729th base (allele A), 43574745th base (allele G), 43574800th base (allele C), 43574824th base (allele G), 43574838th base (allele T), 43574849th base (allele T), 43574860th base (allele C), 43574861st base (allele A), 43574875th base (allele C), 43574878th base (allele G), 43574881st base (allele C), 43574882nd base (allele G), 43574901st base (allele C), 43574929th base (allele A), 43574962nd base (allele G), 43575013th base (allele A), 43575019th base (allele C), 43575025th base (mutation from AT to allele A), 43577064th base (allele C), 43577122nd base (mutation from CCT to allele C), 43577151st base (allele C), 43577166th base (allele T), 43578565th base (allele G), 43578577th base (allele T), 43578583rd base (allele T), 43581834th base (allele A), 43586411th base (allele T), 43586884th base (allele C), 43586893rd base (allele C), 43586897th base (allele G), 43586909th base (allele C), 43586941st base (allele C), 43586965th base (allele G), 43590824th base (allele G), 43590888th base (allele G), 43590947th base (allele C), 43590951st base (mutation from CAATCTAT to allele C), 43591954th base (allele C), 43591986th base (allele A), 43591998th base (allele A), 43592040th base (allele T), 43592328th base (allele G), 43592335th base (allele T), 43592390th base (allele T), 43625187th base (allele C), 43625278th base (allele A) and 43818077th base (allele C) in chromosome 2 of GRCH37/hg19.

5. A method for providing information to predict sulfonylurea dependency in diabetes mellitus patients, comprising identifying at least one of the following:
A first mutation selected from the group consisting of mutations of the 43801878th base (allele A), the 43801909th base (allele T), the 43776463rd base (allele C), the 43625187th base (allele C), the 43520160th base (allele C), and the 43805692nd base (allele G) in chromosome 2 of GRCH37/hg19 THADA (THADA armadillo repeat containing); or
A second mutation that reduces the function of the THADA protein and has an allele frequency of 5% or less.

6. The method of claim 5, wherein the second mutation is a missense mutation, frameshift mutation, nonsense mutation, or splice site mutation.

7. The method of claim 5, wherein the agent is a primer or probe that specifically binds to the THADA gene.

8. The method of claim 5, wherein the second mutation is selected from the group consisting of: mutations of the 43230964th base (allele C), 43231141st base (allele A), 43231295th base (allele T), 43231306th base (allele A), 43231306th base (allele C), 43231323rd base (allele T), 43232797th base (allele G), 43232820th base (allele A), 43232850th base (allele T), 43232874th base (allele C), 43232880th base (mutation from ACT to allele A), 43279821st base (allele A), 43279824th base (allele C), 43279840th base (allele T), 43279855th base (allele C), 43279867th base (allele G), 43279875th base (allele G), 43286968th base (allele T), 43286970th base (allele C), 43286995th base (allele A), 43286998th base (allele T), 43287058th base (allele C), 43291749th base (allele A), 43292125th base (allele G), 43292130th base (allele T), 43292144th base (allele T), 43292201st base (allele C), 43292983rd base (allele A), 43293195th base (allele G), 43320475th base (allele G), 43344122nd base (allele G), 43344138th base (allele A), 43397971st base (allele G), 43397976th base (allele C), 43398003rd base (allele A), 43398005th base (allele T), 43398030th base (allele A), 43398105th base (allele C), 43428128th base (allele C), 43428130th base (allele A), 43428131st base (allele A), 43428134th base (allele C), 43428194th base (allele C), 43430242nd base (allele A), 43430259th base (allele A), 43430261st base (allele C), 43430265th base (allele T), 43430277th base (allele A), 43430288th base (allele C), 43485283rd base (allele C), 43485286th base (allele C), 43485321st base (allele T), 43485322nd base (allele A), 43498843rd base (allele A), 43498867th base (allele A), 43498868th base (allele A), 43498876th base (allele A), 43498877th base (allele G), 43498912th base (allele T), 43498913th base (allele A), 43498915th base (allele A), 43498930th base (allele A), 43498937th base (allele A), 43498941st base (mutation from AT to allele A), 43505722nd base (allele A), 43508647th base (allele A), 43508653rd base (allele C), 43508657th base (allele C), 43508676th base (allele T), 43508677th base (allele C), 43508683rd base (allele C), 43508688th base (allele T), 43508710th base (allele C), 43508751st base (allele A), 43520160th base (allele C), 43527891st base (allele C), 43527894th base (allele T), 43527898th base (allele A), 43527934th base (allele T), 43527966th base (allele G), 43527970th base (allele C), 43541167th base (allele T), 43541237th base (allele A), 43541254th base (allele C), 43541296th base (allele T), 43541298th base (mutation from CAT to allele C), 43549266th base (allele C), 43549356th base (allele T), 43549363rd base (allele T), 43551812th base (allele G), 43551815th base (allele T), 43551906th base (allele T), 43552202nd base (allele T), 43552203rd base (allele G), 43552203rd base (allele G), 43552246th base (allele T), 43552255th base (allele G), 43552256th base (allele C), 43552279th base (allele G), 43552285th base (allele A), 43552311th base (allele G), 43552312th base (allele G), 43552316th base (allele A), 43556356th base (allele C), 43556467th base (allele G), 43556467th base (allele T), 43556495th base (allele A), 43556522nd base (allele T), 43560299th base (allele A), 43560334th base (allele C), 43560359th base (allele A), 43566712th base (allele C), 43566745th base (allele A), 43566760th base (allele A), 43566766th base (allele T), 43566785th base (allele T), 43566791st base (allele G), 43566808th base (allele A), 43570390th base (allele C), 43570392nd base (allele C), 43570471st base (allele G), 43570490th base (allele G), 43571736th base (allele A), 43571736th base (mutation from G to allele GCA), 43571742nd base (allele T), 43571757th base (allele C), 43571759th base (allele T), 43571760th base (allele C), 43571762nd base (allele C), 43571766th base (allele G), 43571775th base (allele G), 43571804th base (allele A), 43571822nd base (allele T), 43571858th base (allele T), 43572900th base (allele T), 43572911th base (allele A), 43572949th base (allele G), 43574347th base (allele A), 43574378th base (allele C), 43574429th base (allele C), 43574432nd base (allele T), 43574440th base (allele A), 43574473rd base (allele T), 43574474th base (allele C), 43574477th base (allele A), 43574551st base (allele G), 43574606th base (allele G), 43574690th base (allele G), 43574693rd base (allele A), 43574714th base (allele A), 43574729th base (allele A), 43574745th base (allele G), 43574800th base (allele C), 43574824th base (allele G), 43574838th base (allele T), 43574849th base (allele T), 43574860th base (allele C), 43574861st base (allele A), 43574875th base (allele C), 43574878th base (allele G), 43574881st base (allele C), 43574882nd base (allele G), 43574901st base (allele C), 43574929th base (allele A), 43574962nd base (allele G), 43575013th base (allele A), 43575019th base (allele C), 43575025th base (mutation from AT to allele A), 43577064th base (allele C), 43577122nd base (mutation from CCT to allele C), 43577151st base (allele C), 43577166th base (allele T), 43578565th base (allele G), 43578577th base (allele T), 43578583rd base (allele T), 43581834th base (allele A), 43586411th base (allele T), 43586884th base (allele C), 43586893rd base (allele C), 43586897th base (allele G), 43586909th base (allele C), 43586941st base (allele C), 43586965th base (allele G), 43590824th base (allele G), 43590888th base (allele G), 43590947th base (allele C), 43590951st base (mutation from CAATCTAT to allele C), 43591954th base (allele C), 43591986th base (allele A), 43591998th base (allele A), 43592040th base (allele T), 43592328th base (allele G), 43592335th base (allele T), 43592390th base (allele T), 43625187th base (allele C), 43625278th base (allele A) and 43818077th base (allele C) in chromosome 2 of GRCH37/hg19.

9. The method of claim 5, further comprising providing information that an individual possessing the first or second mutation exhibits sulfonylurea dependency.
